(19) United States
(12) Patent Application Publication
(10) Pub. No.: US 2006/0004281 A1
(43) Pub. Date: Jan. 5, 2006

(54) Vest-based respiration monitoring system

(76) Inventor: Michael Saracen, Oakland, CA (US)

Correspondence Address: BLAKELY SOKOLOFF TAYLOR & ZAFMAN, 12400 WILSHIRE BOULEVARD, SEVENTH FLOOR, LOS ANGELES, CA, 90025-1030, US

(21) Appl. No.: 11/172,598

(22) Filed: Jun. 29, 2005

Related U.S. Application Data
(60) Provisional application No. 60/584,304, filed on Jun. 30, 2004.

(51) Int. Cl. A61B 5/05

(52) U.S. Cl. 600/414

(57) ABSTRACT

An apparatus and method to track the movement of a pathological anatomy in real-time during radiation treatment.

This application claims the benefit of U.S. Provisional Patent Application No. 60/584,304 entitled “VEST-BASED RESPIRATION MONITORING SYSTEM,” filed Jun. 30, 2004, the contents of which are incorporated by reference herein.

### TECHNICAL FIELD

This invention relates to the field of radiation treatment, and in particular, to a system of tracking the movement of a pathological anatomy during respiration.

### BACKGROUND

Tumors and lesions are types of pathological anatomies characterized by abnormal growth of tissue resulting from the uncontrolled, progressive multiplication of cells, while serving no physiological function. As an alternative to invasive surgery, pathological anatomies can now be treated non-invasively, for example, by external beam radiation therapy. In one type of external beam radiation therapy, an external radiation source is used to direct a sequence of x-ray beams at a tumor site from multiple angles, with the patient positioned so the tumor is at the center of rotation (isocenter) of the beam. As the angle of the radiation source is changed, every beam passes through the tumor site, but passes through a different area of healthy tissue on its way to the tumor. As a result, the cumulative radiation dose at the tumor is high and the average radiation dose to healthy tissue is low. The term radiotherapy refers to a procedure in which radiation is applied to a target region for therapeutic, rather than necrotic, purposes. The amount of radiation utilized in radiotherapy treatment sessions is typically about an order of magnitude smaller, as compared to the amount used in a radiosurgery session. Radiotherapy is typically characterized by a low dose per treatment (e.g., 100-200 centi-Grays (cGy)), short treatment times (e.g., 10 to 30 minutes per treatment) and hyperfractionation (e.g., 30 to 45 days of treatment). For convenience, the term “radiation treatment” is used herein to mean radiosurgery and/or radiotherapy unless otherwise noted by the magnitude of the radiation.

One challenge facing the delivery of radiation to treat pathological anatomies is identifying the target (i.e. tumor location within a patient). The most common technique currently used to identify and target a tumor location for treatment involves a diagnostic x-ray or fluoroscopy system to image the patient's body to detect the position of the tumor. This technique assumes that the tumor is stationary. Even if a patient is kept motionless, radiation treatment requires additional methods to account for movement due to respiration, in particular when treating a tumor located near the lungs. Breath hold and respiratory gating are two primary methods used to compensate for target movement during respiration while a patient is receiving conventional radiation treatments.

Breath hold requires the patient to hold their breath at the same point in their breathing cycle and only treats the tumor when the tumor is stationary. A respirometer is often used to measure the tidal volume and ensure the breath is being held at the same location in the breathing cycle during each irradiation. This method takes longer than a standard treatment and often requires training the patient to hold their breath in a repeatable manner.

Respiratory gating is the process of turning on the radiation beam as a function of a patient's breathing cycle. When using a respiratory gating technique, treatment is synchronized to the individual's breathing pattern, limiting the radiation beam delivery to only one specific part of the breathing cycle and targeting the tumor only when it is in the optimum range. This treatment method may be much quicker than the breath hold method but requires the patient to have many sessions of training to breath in the same manner for long periods of time. This training requires many days of practice before treatment can begin. This system may also require healthy tissue to be irradiated before and after the tumor passes into view to ensure complete coverage of the tumor. This can add an additional margin of 5-10 mm on top of the margin normally used during treatment.

Attempts have been made to avoid the burdens placed on a patient from breath hold and respiratory gating techniques. In another method to track the movement of a tumor in real time during respiration, a combination of internal imaging markers and external position markers has been used to detect the movement of a tumor. In particular, fiducial markers are placed near a tumor to monitor the tumor location. The position of the fiducial markers is coordinated with the external position markers to track the movement of the tumor during respiration. External position markers are used because the fiducial markers are typically monitored with x-ray imaging. Because it may be unsafe to expose the patient continuously to x-rays to monitor the fiducials, the position of the markers can be used to predict the position of the fiducial markers between the longer periods of x-ray images. One type of external position markers integrates light emitting diodes (LEDs) into a vest that is worn by the patient. The flashing LEDs are detected by a camera system to track movement. FIG. 1 illustrates a typical configuration of a real time tracking vest for radiation treatment. The LEDs are positioned on the vest while the fiducials are planted within the patient near the tumor. One problem with LED-based vests is that in the internal images, typically generated by x-ray imaging, to display the position of the fiducial marker, the wires routed along the vests for the LEDs are also displayed. The wiring for the LEDs are metallic (typically copper wire) so the x-ray imaging detects both the fiducial marker and the wiring making the two not easily discemable. Because the image of the target region containing the tumor cannot be clearly visualized by the operator, planning and executing a successful radiation treatment plan may be difficult.

### BRIEF DESCRIPTION OF THE DRAWINGS

The present invention is illustrated by way of example, and not by way of limitation, in the figures of the accompanying drawings.

FIG. 1 illustrates a typical configuration of a real time tracking vest for radiation treatment.

FIG. 2 illustrates one part of an external motion tracking system for use during radiation treatment.

FIG. 3A is an oblique-front view of a fiber optic beacon.

FIG. 3B is an oblique top view of a fiber optic beacon.

FIG. 3C is a top view of a fiber optic beacon.

FIG. 3D is bottom view of a fiber optic beacon.

FIG. 3E is a side view of a fiber optic beacon.

FIG. 3F is a front view of a fiber optic beacon.

FIG. 3G is perspective side view of a fiber optic beacon.

FIG. 4 illustrates a fiber optic cable bundle having a quick connector.

FIG. 5A shows a front view of a respiration monitoring vest.

FIG. 5B shows a back view of a respiration monitoring vest.

FIG. 6 illustrates one embodiment of a configuration for respiration tracking system during radiation treatment.

FIG. 7 illustrates a cross-sectional view of a chest region during respiration and various elements of the internal imaging and respiration monitoring system working together to track motion of a patient.

FIG. 8 illustrates one configuration of a magnetic motion tracking system.

FIG. 9 illustrates a top view of a patient and showing the position of pathological anatomy that is targeted for radiation treatment.

FIG. 10 illustrates a side view of the magnetic motion tracking configuration for a patient lying treatment couch.

FIG. 11 illustrates respiration monitoring system that includes the use of LED as beacons for motion tracking.

FIG. 12 illustrates another respiration monitoring system that includes the use of LED as beacons for motion tracking.

FIG. 13 is a flowchart describing one method for motion tracking during respiration.

FIG. 14 illustrates one embodiment of systems that may be used to perform radiation treatment in which features of the present invention may be implemented.

FIG. 15 illustrates one embodiment of a treatment delivery system.

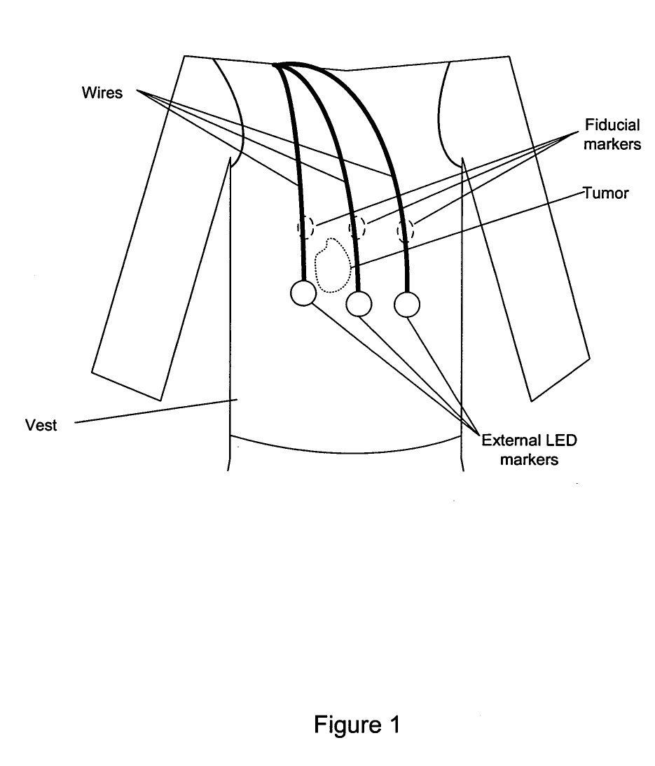

### DETAILED DESCRIPTION

In the following description, numerous specific details are set forth such as examples of specific systems, components, methods, etc. in order to provide a thorough understanding of the present invention. It will be apparent, however, to one skilled in the art that these specific details need not be employed to practice the present invention. In other instances, well-known components or methods have not been described in detail in order to avoid unnecessarily obscuring the present invention. It is understood that the figures herein are not necessarily drawn to scale, and the relative dimensions of the physical structure should not be inferred from the relative dimensions shown in the drawings.

Embodiments of a method and apparatus to track the movement of a pathological anatomy during respiration are described. In one embodiment, a combination of a first reference object placed outside of a patient and a second reference object placed within a patient (e.g., fiducial markers) is used to track the movement of a pathological anatomy during respiration. The second reference object serves as a marker for an internal image of a treatment area that includes the pathological anatomy. The position of the first reference object and the second reference object can be correlated to determine a position of pathological anatomy in real-time. By accurately and clearly following the movement of the pathological anatomy in real-time, a radiation treatment can be performed while a patient is breathing normally. One advantage of the motion tracking system described herein is that the first reference object does not appear in the internal image of the treatment area, thereby avoiding any confusion as to the identity and location of the second reference object. In one embodiment, an internal imaging system and an external motion tracking system are integrated with a radiation treatment system to track movement of a pathological anatomy (in real-time or near real-time) during radiation treatment.

The term “real-time” refers to a time scale that is substantially simultaneous to the actual radiation treatment delivery session, for example, at a rate approximately equal to or greater than 1 Hertz (Hz). The term “near real-time” refers to a time scale that is slower than real-time, for example, by about one or more orders of magnitude less than the time scale of real-time. As an example, the time scale for acquiring x-ray images, which may range from about a fraction of a second to about several seconds may be considered near real-time.

According to one aspect of the present invention, the external motion tracking system generally includes a reference object adapted to be tightly attached to a patient's upper body and a tracking system for tracking the movement of the reference object in real-time. The tracking system includes a detecting system for detecting the position of the reference object and a computer system for control of the detecting system and/or the reference object, and for calculating the position of the reference object in a coordinate system. The movement of the reference object is correlated with the respiratory movement of the patient. The positions of the reference object recorded by the tracking system are transmitted to a treatment system, which uses this information to determine a real-time position of the treatment target, for example a pathological anatomy. The treatment system then synchronizes the treatment to the moving pathological anatomy, thus enabling the radiation treatment system to administer treatment to the pathological anatomy in a dynamical and highly accurate manner.

In one embodiment, fiducial markers are used for the internal imaging of a pathological anatomy and the reference object is part of an external tracking system that includes a vest to be worn by the patient during radiation treatment. In one embodiment, the reference object is a fiber optic beacon disposed on the vest and connected to a light emitting source via a fiber optic cable. The light emitted from the beacon through the fiber optic cable is detected by a camera system to track movement. No element of the fiber optic system (e.g., fiber optic cable, fiber optic beacon) is displayed on the internal image of a treatment area, because substantially no metallic materials are part of the fiber optic system. The only markers displayed on the internal image generated by x-ray imaging are the fiducial markers, which track the position of the pathological anatomy. If elements of both the fiber optic system and the fiducial markers were displayed on the internal image, it may not be possible to distinguish between the two easily.

In an alternative embodiment, the reference object is a magnetic sensor disposed on the vest, with the patient wearing the vest and placed in an electromagnetic field created by a transmitter. The magnetic sensor is connected to a digital processing system that can determine the position and orientation of the patient. Similar to the fiber optic system, elements of a magnetic sensor system is not displayed on the internal image generated by x-ray imaging. In yet another embodiment, the reference object can be an LED beacon having a connecting wire that is thin enough such that the wire is easily discernable from the fiducial marker. Alternatively, the layout of the wire leading to an LED can be configured as so that there is no overlap with the fiducial marker. For example, the wire is positioned so that it does not rest directly over a fiducial marker planted near a target region within the patient.

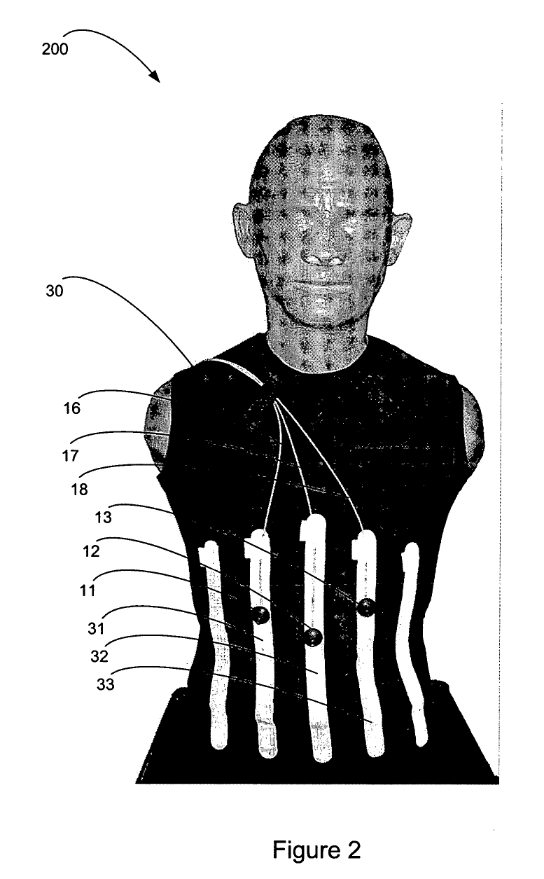

FIG. 2 illustrates one part of an external motion tracking system 200 for use during radiation treatment. Tracking system 200 includes a vest 30 that is tightly secured to the torso of the patient as shown. FIG. 2 illustrates a front view of vest 30, which in one embodiment, is made from material such as spandex, lycra, and the like that conforms to the shape of the patient's body without pleat or spacing between the patient's body and vest 30. One or more fiber optic beacons (e.g., beacon 11, beacon 12, and beacon 13) are secured to an outer surface of vest 30. In one embodiment, the fiber optic beacons are secured to vest 30 with one or strips of small hooks or loops such as strip 31, strip 32, and strip 33. The strips may be include any type of hook and loop fasteners known in the art, for example VELCRO® strips made by Velcro Industries B.V. For example, a bottom surface of fiber optic beacon 11 may include a series of hooks and strip may include a series of loops to secure fiber optic beacon 11 to vest 30. Each fiber optic beacon is connected to a light source with a fiber optic cable as shown. Beacon 11 is connected via cable 16, beacon 32 is connected via cable 17, and beacon 13 is connected via cable 18. In one embodiment, the fiber optic cables, beacons and vest 30 are made from plastic or other types of polymers exhibiting material properties of plastic, thereby eliminating metal from the treatment field, so that the x-ray system can clearly image any implanted fiducials with no artifacts created during a CT scan around the radiation treatment region. In one particular embodiment, fiber optic beacons 11-13, fiber optic cables 16-18, and vest 30 are made from a radiolucent material to allow for artifact free x-ray images during radiation treatment. Vest 30 of FIG. 2 has been illustrated and described with three fiber optic beacons, but the external motion tracking system is not limited to three fiber optic beacons. In alternative embodiments, vest 30 may include less than three or more than three fiber optic beacons.

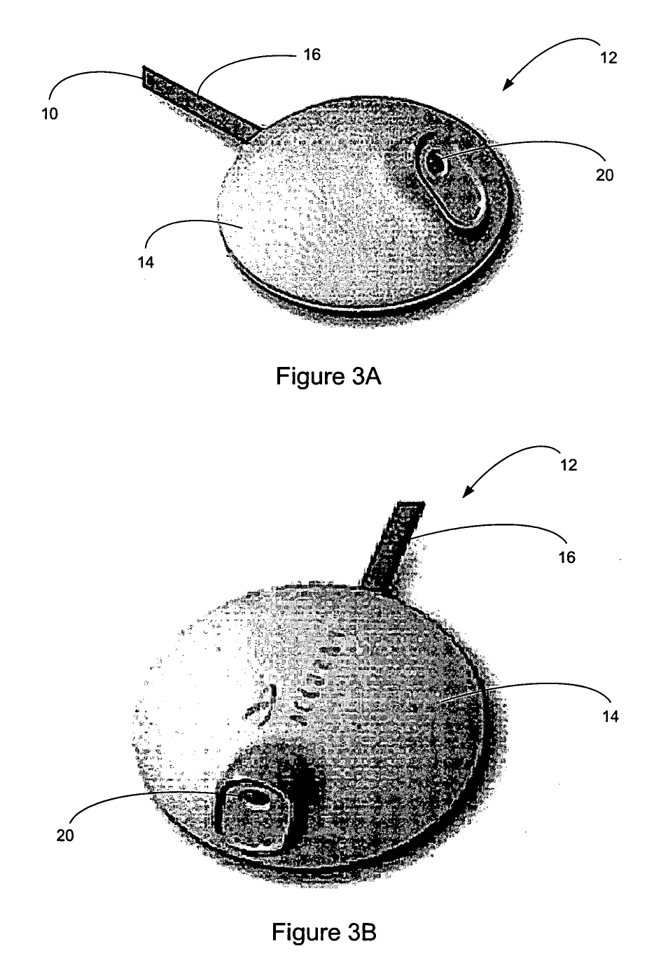

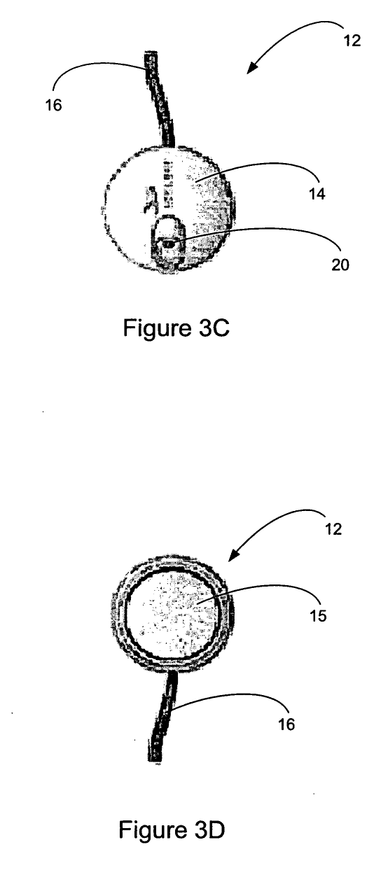

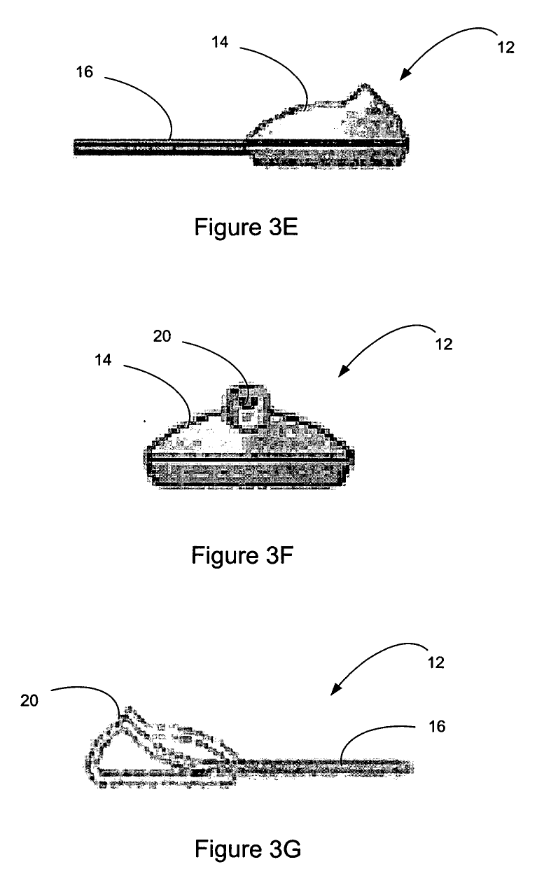

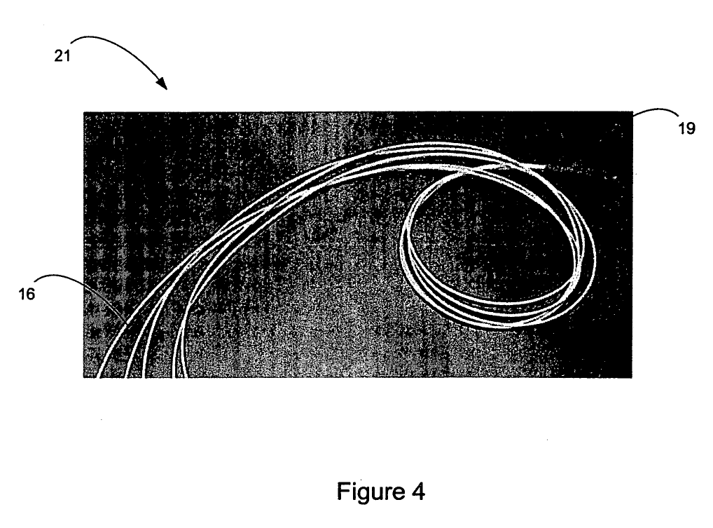

FIGS. 3A-3G illustrate different views of fiber optic beacon 12. As the three fiber optic beacons are substantially similar in form and function, a detailed description is provided with respect to one fiber optic beacon for clarity. FIG. 3A is an oblique-front view and FIG. 3B is an oblique top view of fiber optic beacon 12. FIG. 3C is a top view and FIG. 3D is bottom view of fiber optic beacon 12. FIG. 3E is a side view, FIG. 3F is a front view, and FIG. 3G is perspective side view of fiber optic beacon 12. As shown in the figures, the fiber optic beacon 12 is constructed with a dome shape. An elongated fiber optic cable 16 extends between two ends, a first end 10 adapted to be connected to a breakout box (not shown), and a second end 20 secured to beacon 12 and extending through beacon 12 to at least an outer surface 14, as illustrated in FIGS. 3A, 3B, and 3G. As shown in FIG. 3D, the bottom surface 15 of the beacon 12 is provided with a piece of fabric of small hook and loop fasteners. The portion near the second end 20 of the fiber optic cable 16 is received in a channel defined in the beacon 12, as shown in the perspective side view in FIG. 3G. The angle of the fiber 16 with respect to the bottom surface 15 of the beacon 12 is about 15 degrees in order to provide better visualization of the light emitted from the second end 20 by the eyes of a user or an external detector. In the embodiment having three fiber optic beacons 12 (e.g., as illustrated in FIG. 2), three fiber optics 16-18 coupled to fiber optic beacons 11-13 respectively. As illustrated in FIG. 4, each fiber optic cable (e.g., fiber optic cable 16) of a cable bundle 21 is joined by a quick connector 19 near the first end (e.g., first end 10), so that quick connector can be used to connect all the fiber optic cables with the breakout box at once without the need for individually connecting each fiber optic cable.

In one embodiment, the breakout box houses light emitting diodes (LEDs), which are associated with the first end 18 of the fiber optic cable 16. The LEDs emit light, for example in the red spectrum, and transmits the light to the first end 18 of fiber optic cable 16. The light is then propagated through fiber optic cable 16 and emitted out of the second end 20 of fiber optic beacon 12.

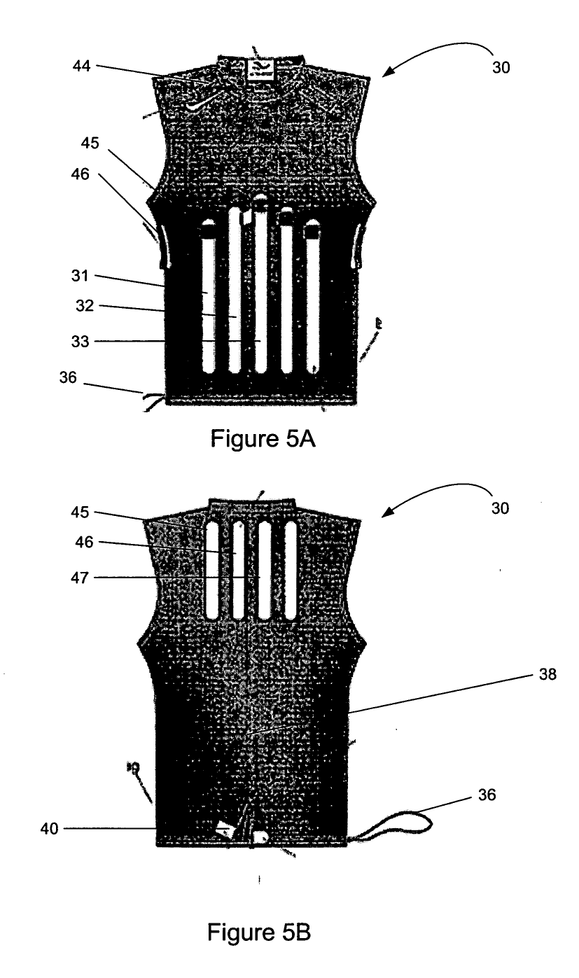

In one embodiment, the respiration monitoring system includes a vest 30 which is customized to fit a particular patient tightly. FIG. 5A shows a front view of the vest 30 and FIG. 5B shows a back view of the vest 30. The vest 30 is made from a tight fitting material, for example, spandex, lycra, and the like, that conforms to the shape of the patient's body without pleat or spacing between the patient's body and the vest. The vest includes a mechanism for securing the reference object, e.g., the fiber optic beacon 12, thereon. In the exemplary embodiment shown in FIGS. 5A and 5B, vest 30 is provided with strips of fabric of small hooks or loops, for example, VELCRO® strips 32, at both the front side and the back side of the vest 30 for both prone and supine treatments. The bottom surface of the fiber optic beacon 12 is also provided with a piece of fabric of small hooks or loops to engage with the strips 32 on the vest 30. In use, the fiber optic beacon 12 can be easily attached to the vest 30, and after use, can be easily removed. Devices other than fabric of small hooks and loops also can be used for securing the beacons 12 to the vest 30. The vest 30 has a waist drawstring 36 passing through a channel formed at the bottom boundary of the vest 30. A traditional drawstring with a stop member that can slide along the drawstring and can stop at any point on the drawstring to fasten the drawstring can be used. The drawstring 36 is used to fasten the bottom boundary of the vest 30 to prevent the vest 30 from rolling up to the patient's upper body.

The vest 30 also includes a closure assembly, which may be an elongated zipper 38, attached on the back of the vest 30 and extending from the neck of the vest 30 to the bottom of the vest 30. The elongated zipper 38 allows easy applications of the vest 30 to the patients with limited mobility. In a particular embodiment, a ribbon tab 40 with small fabric hooks or loops is attached to the sliding head of the zipper 38. The ribbon tab 40 can be attached to a piece of fabric of small hooks or loops 42 at the bottom boundary of the vest 30, so that after the zipper head is slid down to dose the zipper 38, the ribbon tab 40 can be secured at the bottom of the vest 30, thus preventing the zipper head from moving upward to unintentionally open the zipper 38.

The vest 30 may include more ribbon tabs as denoted by number 44 with small hooks or loops at the bottom surface of the ribbon tabs. In use, the ribbon tabs 44 are placed over the fiber optics 16 to facilitate to secure the fiber optics 16 and the beacons 12 on the vest 30. The vest 30 may further include brand tags at the collar and/or at the side seam of the vest 30, as shown in FIGS. 5A and 5B. The vest 30 may further include one or more pocket for containing the patient's ID tag, picture, and/or other documents.

Tracking the motion of a pathological anatomy caused by respiration as described with respect to FIGS. 2-5 may be integrated with a therapeutic radiation treatment system. The therapeutic radiation treatment system is generally associated with an imaging system, for example, an x-ray imaging system for internal imaging of the treatment area. The fiber optic cable 16, fiber optic beacon 12, and the vest 30 are made from plastic, eliminating metal from the treatment field, so that the x-ray imaging system is able to see any implanted fiducials during treatment while no artifacts are created during a CT scan. In one embodiment, the fiber optic cable 16, fiber optic beacon 12, and the vest 30 are made from a radiolucent material to allow for artifact free x-ray images during treatment.

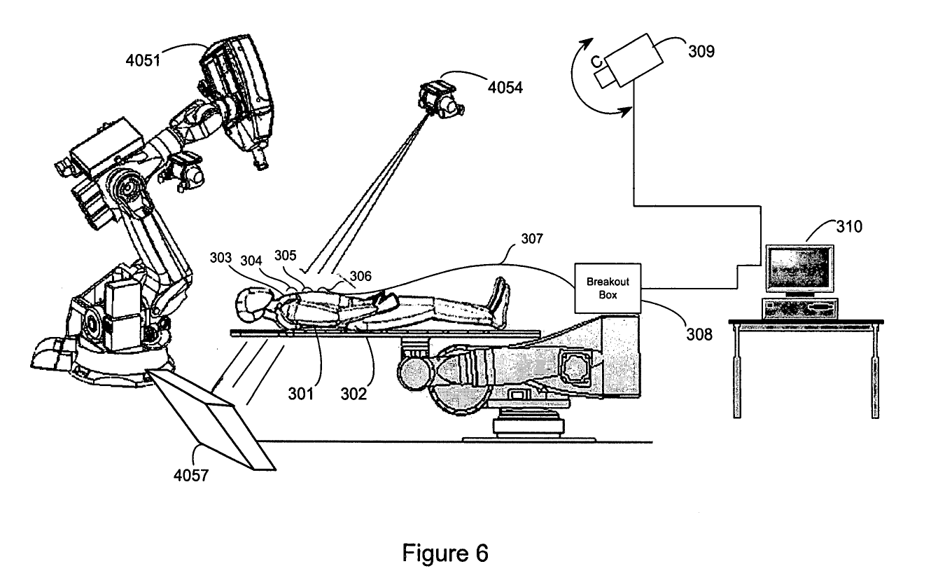

FIG. 6 illustrates one embodiment of a configuration 300 for respiration tracking system during radiation treatment. The respiration tracking system coordinates an internal image of the patient 301 near a pathological anatomy region marked by fiducials (not shown) and an external motion tracking (e.g., the up-and-down movement of the chest during breathing). The respiration tracking system includes vest 303 having one or more fiber optic beacons represented by beacons 304-306. Each beacon is connected by a fiber optic cable (e.g., cable 307), which at an opposite end is connected to a light source such as breakout box 308. The respiration tracking system also includes a real-time image guidance system, which includes a localizer, a digitizer system, and a computer controller. The localizer includes a camera system, which includes one or more cameras 309 working in conjunction with each other, to detect a point source of light from each fiber optic beacon and determine the location of the point source of light in an ordinate system. Breakout box 308 originates the light that propagates through each fiber optic cable and emitted through a beacon. In one embodiment, the light originates from an LED. Each point source of light, which is associated with a beacon (e.g., beacon 304), flashes in a pre-defined sequence so that camera 309 is able to identify each beacon and its orientation. The positions of the light sources are recorded in real-time and transmitted to a treatment system, for example, a therapeutic radiation treatment system, which uses this information to determine the real-time position of the treatment target. A radiation delivery system is then able to synchronize the radiation delivery tool to the movement of the treatment target during the treatment.

A digitizer system is connected to the localizer to convert the image data received from the localizer to digital information, and transmits the digital information to the computer controller. The digitizer system and computer controller can be a combination of software and hardware operating on a digital processing system, such as workstation 310. The computer controller, which is connected to the digitizer system and the localizer, controls the camera system 309. The controller is programmed to determine the real-time positions of fiber optic beacons 304-306 and transmits the position information from the beacons to the treatment system.

The computer controller also controls the flashing rate of the light sources on the fiber optic beacons 304-306 responsive to whether the light sources are in the view of the camera systems 309. For example, if the light sources are seen by the camera systems 309, the light sources flash at the rate used for tracking the positions of the light sources, and if any of the light sources on fiber optic beacons 304-306 are not seen by the camera system 309, the light sources emit continuous red light. By this arrangement, the user can visually identify if the beacon is seen by the camera system 309. If any of the light sources on fiber optic beacons 304-306 are not seen by the camera system 309, the user adjusts the position of fiber optic beacons 304-306 until it starts to flash red light, which indicates that it is being seen by the camera system 309.

In one embodiment, as illustrated in FIG. 6, radiation may be delivered by an image-guided, robotic-based radiation treatment system such as the CyberKnife® system developed by Accuray Incorporated of California. The radiation source may be represented by a linear accelerator (LINAC) 4051 mounted on the end of a robotic arm having multiple (e.g., 5 or more) degrees of freedom in order to position the LINAC 4051 to irradiate a pathological anatomy (target region or volume) with beams delivered from many angles in an operating volume (e.g., a sphere) around the patient. The internal image of the treatment region may be generated by an imaging system that includes one or more x-ray sources and x-ray image detectors, such as x-ray source 4054 and detector 4057. In one embodiment, for example, x-ray source 4054 may be nominally aligned to project imaging x-ray beams through patient 301 from an angular position and aimed through the patient 301 on treatment couch 302 toward detector 4057. In another embodiment, a two x-ray sources may be nominally aligned to project imaging x-ray beams through patient 301 from two different angular positions (e.g., separated by 90 degrees, 45 degrees, etc.) and aimed through the patient on treatment couch 302 toward respective detectors.

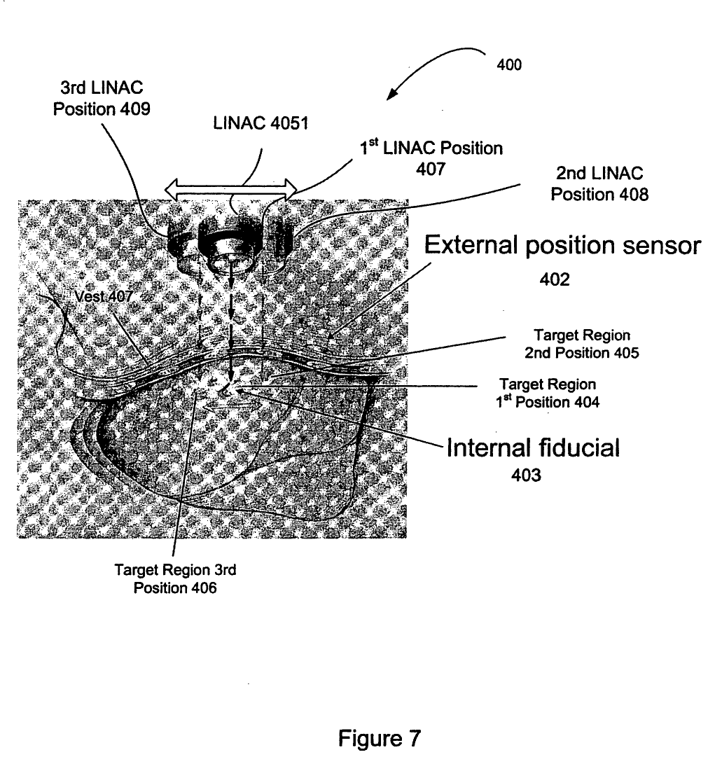

As the pathological anatomy moves with breathing, the LINAC 4051 is able to follow the movement by correlating the signals from fiber optic beacons 303-305 and the internal fiducial marker. FIG. 7 illustrates a cross-sectional view of a chest region 400 during respiration with various elements of the internal imaging and respiration monitoring system working together to track motion of a patient and the treatment region while delivery radiation. The patient is positioned lying flat on a radiation treatment couch (e.g., as illustrated by patient 301 on couch 302). One or more external position sensors, such as fiber optic beacon 402, are disposed on vest 405, which is fitted over the chest area of the patient. The pathological anatomy is designated as part of a target area first position 404 and an internal fiducial marker 403 is positioned near target area first position 404. An x-ray based imaging system is used image target area first position 404 using internal fiducial marker 403.

FIG. 7 illustrates that during respiration, the treatment region containing the pathological anatomy moves between target area first position 404, a second position 405, and a third position 406 relative to internal fiducial marker 403. Beacon 402 detects the motion of the chest area during respiration as it moves between the different positions. This external monitoring is correlated with the internal imaging of target regions 404-406. In this manner, the position of target regions 404-406 can be constantly updated and LINAC 4051 moves with respect to the movement of the target regions. For example, LINAC 4051 is in a first position 407 corresponding to target region first position 404. When the target region moves to target region second position 405, LINAC 4051 moves to second position 408. Similarly, when the target region moves to third target region third position 406, LINAC 4051 moves to third position 409. Radiation delivery may involve beam paths with a single isocenter (point of convergence), multiple isocenters, or with a non-isocentric approach (i.e., the beams need only intersect with the pathological target volume and do not necessarily converge on a single point, or isocenter, within the target).

In one method to integrate the monitoring of motion of patient due to respiration, a preoperative process and a treatment process are involved. In the preoperative process, the patient is provided with a vest 30 that tightly fits the patient's torso area. A picture of the patient is taken and is placed in the pouch on the vest 30 to identify the patient. The patient puts on the vest 30, and the vest 30 is zipped up, and the drawstring 36 is adjusted at the waist for proper fit. The patient is then placed in an immobilization device, which is generated for the patient for holding the patient. The immobilization device, made from a moldable material, is customized to fit with the patient's body curve with the vest on the patient body. The immobilization device is attached to the patient positioning system, holding the patient tightly and preventing the patient from moving during treatment. The patient held in the immobilization device is scanned, typically by a CT scanner, to determine the position of the treatment target. The vest 30, the immobilization device, and the image data are then ready for use in the treatment, which can be conducted after the preoperative process or on another day. In an alternative embodiment, the preoperative process may be performed without the immobilization device.

During treatment, the vest 30 is put on the patient and the patient is immobilized in a treatment position on the patient positioning device. The first end of a fiber optic cable (e.g., first end 10 of fiber optic cable 16) is snapped into a breakout box (e.g., breakout box 308 and connected to the LEDs). The second end of the fiber optic cable (e.g., second end 20) is connected to a fiber optic beacon (e.g., fiber optic beacon 12) and placed on the vest 30. Once the first ends of all the fiber optic cables, which may correspond to the number of beacons placed on vest 30, are connected to the breakout box, the user should be able to see read light emitting out of the beacons from the second ends of the fiber optic cables. The fiber optic beacons (e.g., 12) are placed on the vest 30 on the area of the patient that moves the most with respiration, e.g. the area at or near the diaphragm of the patient. The small hooks or loops on the bottom surface of the beacons engage with the hook or loop fabric strips (e.g., 32) on the vest 30, and thus, allowing each fiber optic beacon to be attached to the vest 30 securely. The beacons are positioned and orientated so that the emitted light can be seen by the camera system (e.g., camera 309). In one embodiment, if the emitted light is a flashing red light, then it is detected by the camera system. If the emitted light is a continuous red light, then it is not detected by the camera system, and the user has to adjust the positions and orientations of the beacons or the camera to make the emitted light from the beacons flash. Once the beacons are aligned to the correct positions, the ribbon tabs (e.g., tab 44) are placed over the fiber optic cables to prevent unwanted movement of the fiber optic cables during the treatment.

During treatment, the camera system 309 and the computer controller track the movement of the beacons 12 with the respiration of the patient, and send the information to the treatment system, so that the treatment system can determine the real-time position of the treatment target and synchronize the movement of the treatment target. Thus the treatment system administers treatment to the treatment target in a dynamic and spatially accurate manner.

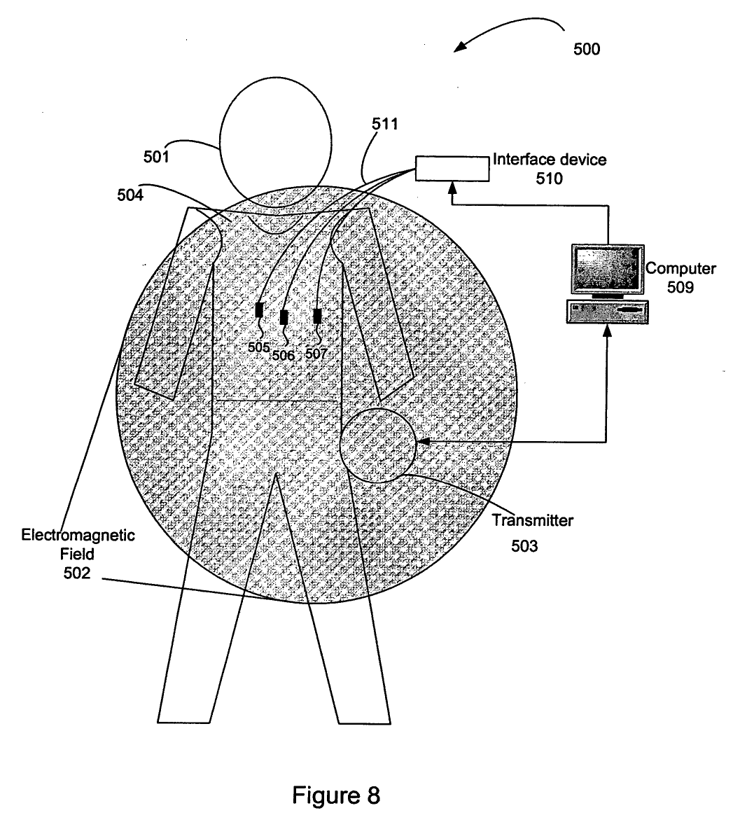

FIGS. 8-10 illustrate another embodiment of a respiration motion tracking system, in which a magnetic-based monitor is correlated with an internal imaging system during radiation treatment. In magnetic tracking, a transmitter broadcasts an electromagnetic field and sensors placed within the magnetic field can capture translation (x, y, z) coordinates and yaw, pitch, roll (y, p, r) rotation coordinates of objects to which the sensors are attached. FIG. 8 illustrates one configuration of a magnetic motion tracking system established for patient 501 viewed from a top position as if patient 501 were lying on a treatment couch. A vest 504, placed on patient 501, includes three magnetic sensors or transducers 505, 506, and 507. Magnetic sensors 505, 506, and 507 are connected to an interface device 510 via wires 511. Interface device 510 is also connected to a digital processing system such as computer 509. A transmitter 503 is also connected to computer 509.

Transmitter 503 creates electromagnetic field 502 within the vicinity of patient 501, and in particular, magnetic sensors 505, 506, and 507 disposed on vest 504. In one embodiment, transmitter 503 includes one or more coils on an orthogonal axes and current (either alternating or direct) is passed through the coils to generate electromagnetic field 502. Magnetic sensors 505, 506, and 507 also include similar coils, but are passive coils that only detect current. The movement of magnetic sensors 505, 506, and 507 during respiration sends a combination of signal strengths to interface 510 which is then interpreted by computer 509 to determine the exact position and orientation of the chest region of patient 501. Interface 510 also serves to filter the signals from magnetic sensors 505, 506, and 507 to reduce jitter. Magnetic transmitter and sensors are known in the art; accordingly, a detailed description is not provided herein.

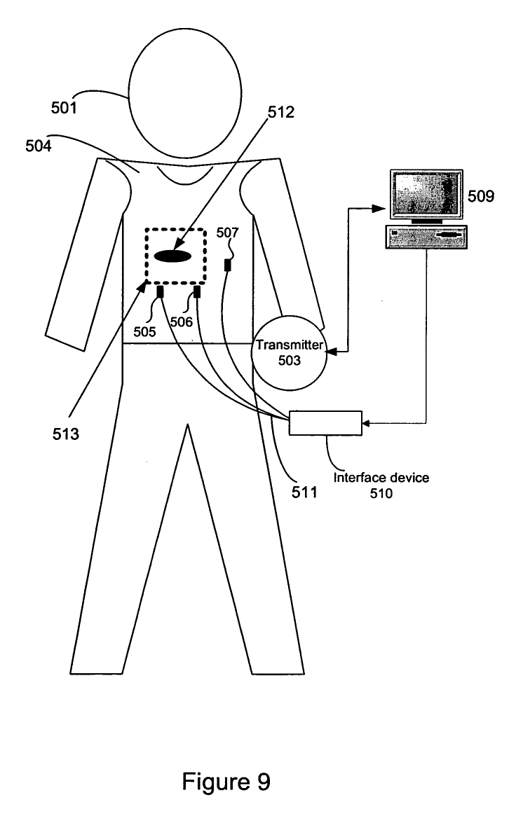

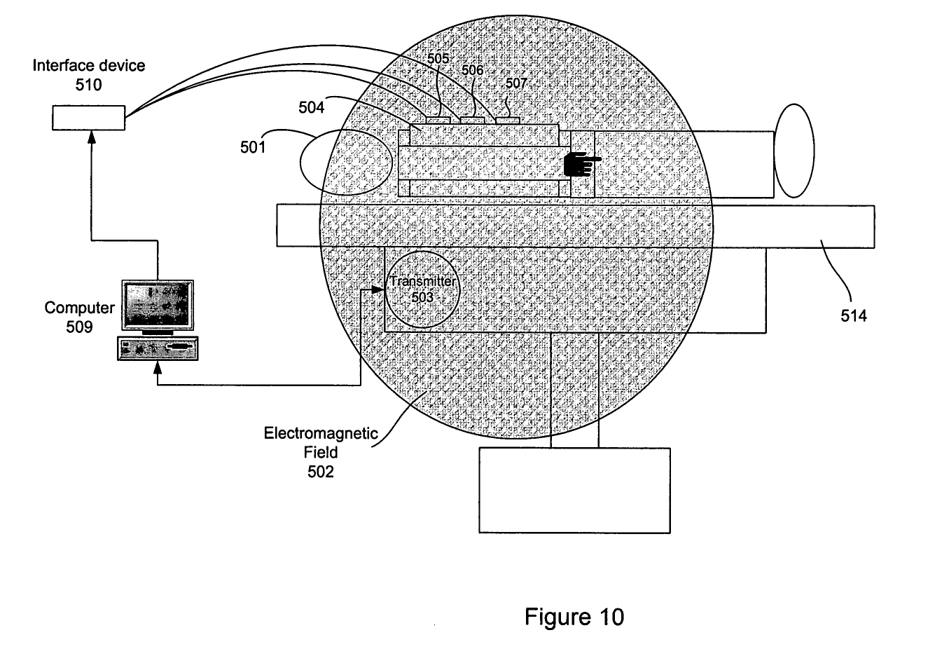

FIG. 9 illustrates another top view of patient 501 and showing the position of pathological anatomy 512 that is targeted for radiation treatment. The region containing and around pathological anatomy 512 is also the internal image zone 513 that is captured in real time during radiation treatment. In one embodiment, internal image zone 513 may include internal fiducial markers (not shown) utilized by an x-ray imaging system. FIG. 10 illustrates a side view of the magnetic motion tracking configuration for patient 501 lying treatment couch 514. In one embodiment, transmitter 503 may be coupled to treatment couch 514 to generate the electromagnetic field 502 that surrounds magnetic sensors 505, 506, and 507 disposed on vest 504. Transmitter 503 does not need to be attached to treatment couch 514, and in alternative embodiments, transmitter 503 is positioned close enough to patient 501 to generate magnetic field 502.

Interface device 510 receives signals from magnetic sensors 505, 506, and 507 corresponding to motion during respiration. The signals are translated and filtered to reduce jitter before being transmitted to computer 509. The motion tracking data provided by interface device 510 is correlated with imaging data of the pathological anatomy 512 (e.g., x-ray imaging of zone 513 with fiducial markers) to provide real-time tracking. For clarity, FIG. 10 is shown without an imaging system and a radiation treatment system such as x-ray source 4054, detector 4057, and LINAC 4051 as described above and shown in FIG. 6. In one embodiment, the magnetic sensor-based tracking system may be integrated with an imaging system and radiation treatment system.

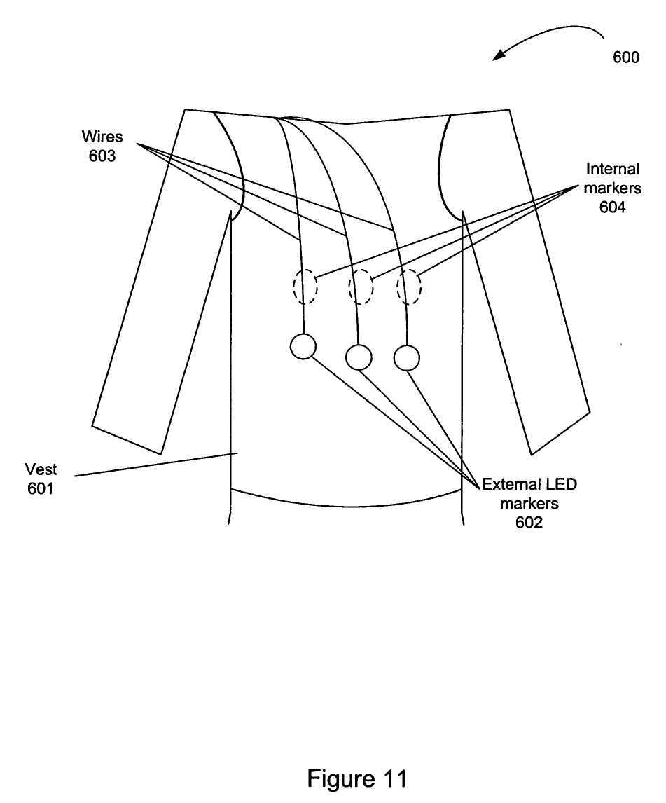

FIG. 11 illustrates an alternative embodiment of a respiration monitoring system 600 that allows for the use of LEDs as reference objects for motion tracking. External LED markers 602 are disposed on vest 601 that are connected to a power source (not shown) by wires 603. One feature in the configuration illustrated in FIG. 11 is that wires 603 are significantly thin enough so that the use of substantially metallic wire to propagate the electrical current to the LEDs, even if they may appear on an internal image of the treatment area, is easily discernable from the internal markers 604 (e.g., fiducials). In one embodiment, a thickness or diameter of each individual wire of wires 603 is less than a width of each internal marker 604. For example, each individual wire of wires 603 may be a single or a bundle of multiple wires woven together to form a greater than 12 gauge wire (i.e., diameter of the wire is less than the diameter of a 12 gauge wire). In one embodiment each individual wire of wires 603 may be greater than a 29 gauge wire. As shown, wires 603 can be disposed directly over an image zone that includes internal markers 604.

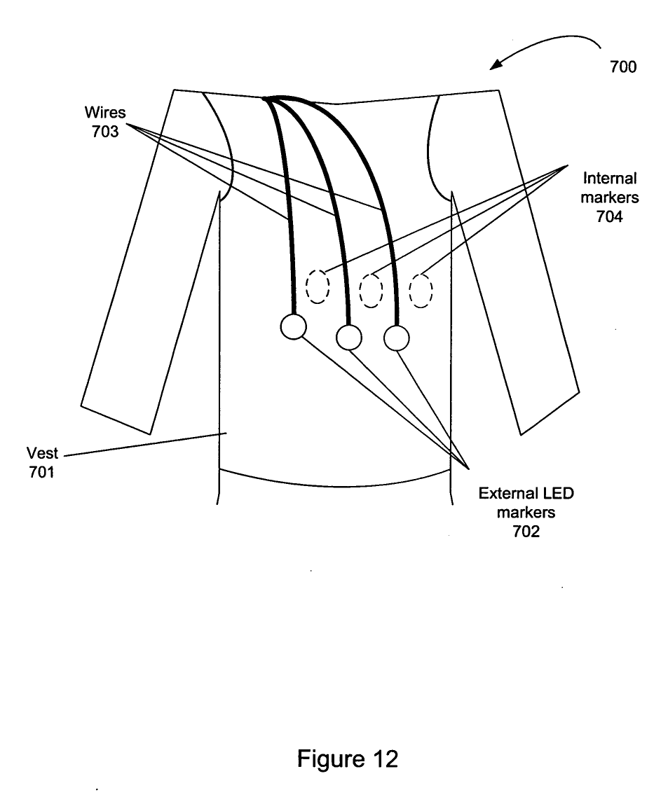

In another embodiment of a respiration monitoring system 700 illustrated in FIG. 12, wires 703 connecting a power source and external LED markers 702 are positioned on vest 701 so that no part of any wire is directly over any of the internal markers 704. The non-overlapping position of wires with respect to internal markers 704 makes the wires easily discernable from the internal markers 704.

Because respiration monitoring system 600 operates similarly to monitoring system 700, a description of both is provided with respected to monitoring system 600. An internal image of the patient near a pathological anatomy region marked by internal markers 604 is correlated with movement caused by respiration as tracked by external LED markers 602. Wires 603 connect LED markers 602 to a power source (such as a breakout box, not shown). In one embodiment, wires 603 may be made of a metallic material, such as copper. The respiration tracking system also includes a real-time image guidance system, which includes a localizer, a digitizer system, and a computer controller. The localizer includes a camera system, which includes one or more cameras (such as camera 309) working in conjunction with each other, to detect a point source of light from each external LED markers 602 and determine the location of the point source of light in an ordinate system. Each of external LED markers 602 flashes in a pre-defined sequence so that the camera is able to identify each marker and its orientation. The positions of the LED markers 602 are recorded in real-time and transmitted to a treatment system, for example, a therapeutic radiation treatment system, which uses this information to determine the real-time position of the treatment target. A radiation delivery system is then able to synchronize the radiation delivery tool to the movement of the treatment target during the treatment.

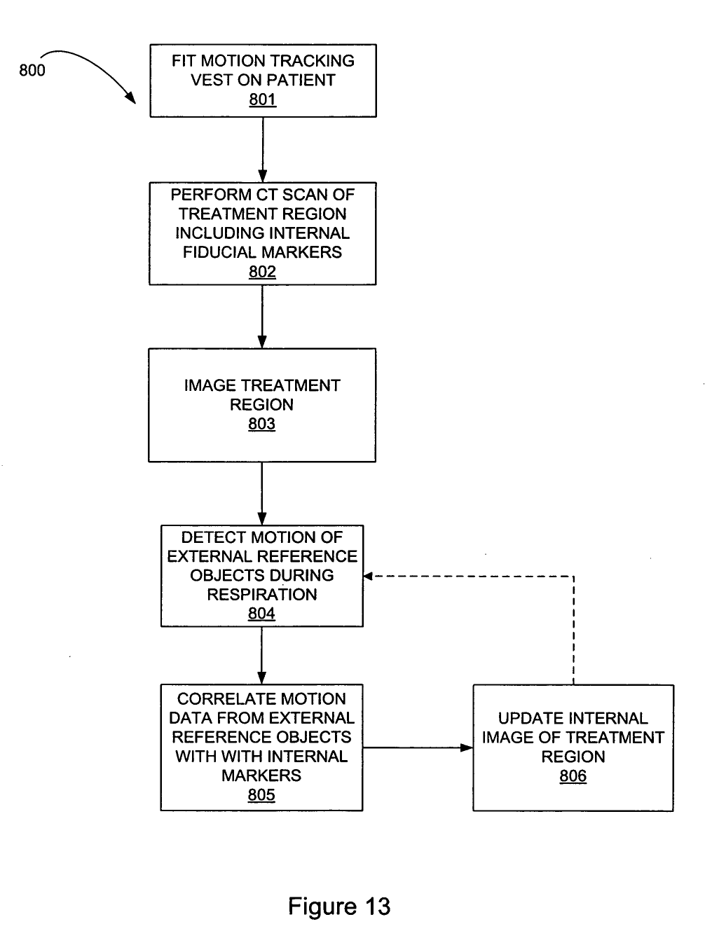

FIG. 13 is a flowchart 800 describing one method for tracking movement of a pathological anatomy caused by respiration. A combination of imaging internal fiducial markers and tracking external reference objects is used to track the movement of the pathological anatomy in real-time, allowing for a radiation delivery source to follow the movement of the pathological anatomy during radiation delivery. The method overcomes the problems of prior art tracking methods because the external reference objects are clearly distinguishable from the internal fiducial markers in the internal image generated by x-ray imaging. Alternatively, the external reference objects do not appear on an internal image generated by x-ray imaging. The external reference object tracking may involve one of fiber optic beacons, magnetic sensors, or LED beacons. The method of flowchart 800 is described with respect to the treatment of a pathological anatomy or tumor located within the chest region of a patient. The patient is first fitted with a motion tracking vest that covers the region under the chest targeted for treatment, step 801. In a first embodiment, the vest may have one or more fiber optic beacons disposed on an outer surface, such as vest 30 and fiber optic beacons 11-13 described above with respect to FIG. 2. In a second embodiment, the vest may have one or more magnetic sensors, such as vest 504 and magnetic sensors 505-507 described above with FIG. 8. In a third embodiment, the vest may have one or more LED markers, such as vest 601 and LED markers 602 described above with respect to FIG. 11.

Next, a CT image is generated of the chest including the target region to determine the position of the pathological anatomy, step 802. The CT image may include the location of one or more fiducial markers near the pathological anatomy. During radiation treatment, the target region is imaged with x-ray imaging and with the aid of the internal fiducial markers (e.g., fiducial 403), step 803. The external reference objects do not appear on the internal image or alternatively, are easily discernable from the fiducial markers. During respiration of a patient, movement of the external reference objects may be detected by a number of different methods, depending on the type of reference object used, step 804. For example, for a fiber optic beacon or an LED marker, an emitted light is detected by a camera system (e.g., camera 309). The fiber optic cables do not appear on the internal images generated by x-ray imaging, allowing the internal fiducial markers to be easily identified. For LED markers, the wires that connect a power source to the LED markers may be made of significantly thin metallic material so that they are easily discemable from the fiducial markers in the internal image (e.g., wires 603 illustrated in FIG. 11). Alternatively, the wires may be positioned on the vest so that there is no overlap with the internal fiducials below the vest (e.g., as illustrated by wires 703 in FIG. 12).

For a magnetic sensor, an electromagnetic field (e.g., electromagnetic field 502) is generated to encompass the patient, and movement of the magnetic sensor is detected by an interface device (e.g., 510) that interprets signals from the magnetic sensor. The electromagnetic field may be generated by a transmitter positioned near the patient or the treatment couch (e.g., as illustrated in FIG. 10). During radiation treatment, the position of the external reference objects is correlated with the internal markers to follow the motion of a pathological anatomy in real-time, step 805. The new position of the target region can then be displayed with an updated internal image, step 806. An internal image of the treatment region can be updated and displayed at regular intervals as movement is detected by the external reference objects (by repeating the process starting at step 804).

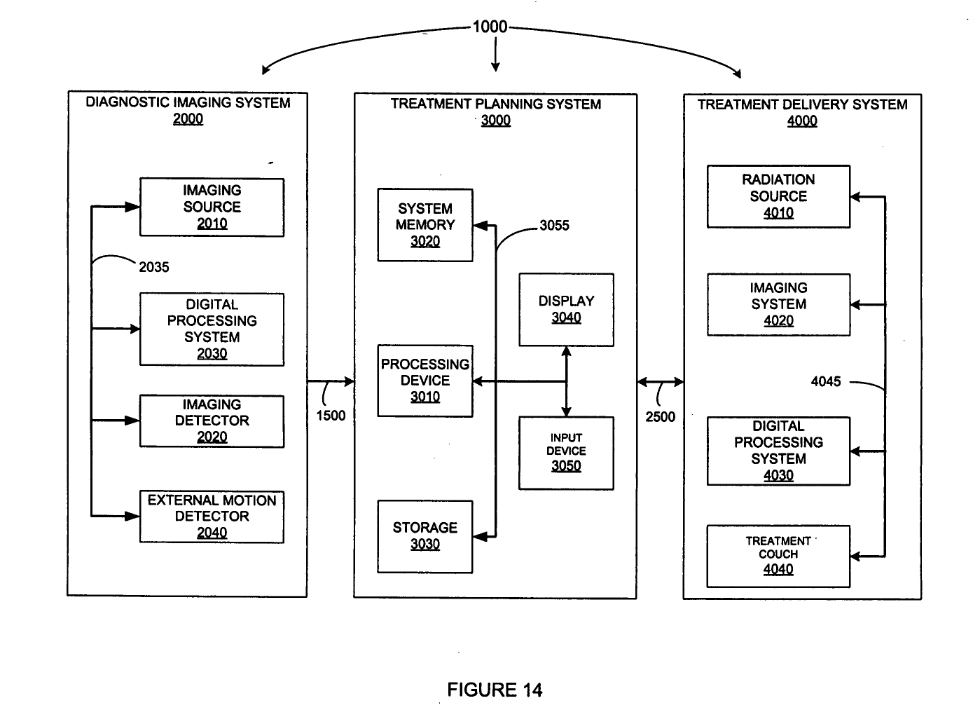

FIG. 14 illustrates one embodiment of systems that may be used to perform radiation treatment in which features of the present invention may be implemented. As described below and illustrated in FIG. 14, system 1000 may include a diagnostic imaging system 2000, a treatment planning system 3000, and a treatment delivery system 4000.

Diagnostic imaging system 2000 may be any system capable of producing medical diagnostic images of a volume of interest (VOI) in a patient that may be used for subsequent medical diagnosis, treatment planning and/or treatment delivery (e.g., image zone 513 shown in FIG. 9). For example, diagnostic imaging system 2000 may be a computed tomography (CT) system, a magnetic resonance imaging (MRI) system, a positron emission tomography (PET) system, an ultrasound system or the like. For ease of discussion, diagnostic imaging system 2000 may be discussed below at times in relation to a CT x-ray imaging modality. However, other imaging modalities such as those above may also be used.

Diagnostic imaging system 2000 includes an imaging source 2010 to generate an imaging beam (e.g., x-rays, ultrasonic waves, radio frequency waves, etc.) and an imaging detector 2020 to detect and receive the beam generated by imaging source 2010, or a secondary beam or emission stimulated by the beam from the imaging source (e.g., in an MRI or PET scan). In one embodiment, diagnostic imaging system 2000 may include two or more diagnostic X-ray sources and two or more corresponding imaging detectors. For example, two x-ray sources may be disposed around a patient to be imaged, fixed at an angular separation from each other (e.g., 90 degrees, 45 degrees, etc.) and aimed through the patient toward (an) imaging detector(s) which may be diametrically opposed to the x-ray sources. A single large imaging detector, or multiple imaging detectors, may also be used that would be illuminated by each x-ray imaging source. For imaging of a target region containing a pathological anatomy, the x-ray source may be aimed toward fiducial markers planted (e.g., fiducial 403) in the patient. Alternatively, other numbers and configurations of imaging sources and imaging detectors may be used.

Diagnostic imaging system 2000 also includes an external motion detector 2040 to monitor the movement of the patient, for example during respiration. The external motion detector 2040 may be a vest-based system that includes fiber optic beacons, magnetic sensors, or LED marker as discussed above. The data from the external motion detector 2040 can be correlated with the imaging detector to track the motion of the pathological anatomy in real time.

The imaging source 2010, the imaging detector 2020, and the external motion detector 2040 are coupled to a digital processing system 2030 to control the imaging operation and process image data. One example of a digital processing system is computer 310 described above with respect FIG. 6. Diagnostic imaging system 2000 includes a bus or other means 2035 for transferring data and commands among digital processing system 2030, imaging source 2010 and imaging detector 2020. Digital processing system 2030 may include one or more general-purpose processors (e.g., a microprocessor), special purpose processor such as a digital signal processor (DSP) or other type of device such as a controller or field programmable gate array (FPGA). Digital processing system 2030 may also include other components (not shown) such as memory, storage devices, network adapters and the like. Digital processing system 2030 may be configured to generate digital diagnostic images in a standard format, such as the DICOM (Digital Imaging and Communications in Medicine) format, for example. In other embodiments, digital processing system 2030 may generate other standard or non-standard digital image formats. Digital processing system 2030 may transmit diagnostic image files (e.g., the aforementioned DICOM formatted files) to treatment planning system 3000 over a data link 1500, which may be, for example, a direct link, a local area network (LAN) link or a wide area network (WAN) link such as the Internet. In addition, the information transferred between systems may either be pulled or pushed across the communication medium connecting the systems, such as in a remote diagnosis or treatment planning configuration. In remote diagnosis or treatment planning, a user may utilize embodiments of the present invention to diagnose or treatment plan despite the existence of a physical separation between the system user and the patient.

Treatment planning system 3000 includes a processing device 3010 to receive and process image data. Processing device 3010 may represent one or more general-purpose processors (e.g., a microprocessor), special purpose processor such as a digital signal processor (DSP) or other type of device such as a controller or field programmable gate array (FPGA). Processing device 3010 may be configured to execute instructions for performing motion tracking operations discussed herein, for example, correlating the movement of a pathological anatomy during respiration.

Treatment planning system 3000 may also include system memory 3020 that may include a random access memory (RAM), or other dynamic storage devices, coupled to processing device 3010 by bus 3055, for storing information and instructions to be executed by processing device 3010. System memory 3020 also may be used for storing temporary variables or other intermediate information during execution of instructions by processing device 3010. System memory 3020 may also include a read only memory (ROM) and/or other static storage device coupled to bus 3055 for storing static information and instructions for processing device 3010.

Treatment planning system 3000 may also include storage device 3030, representing one or more storage devices (e.g., a magnetic disk drive or optical disk drive) coupled to bus 3055 for storing information and instructions. Processing device 3010 may also be coupled to a display device 3040, such as a cathode ray tube (CRT) or liquid crystal display (LCD), for displaying information (e.g., a 2-dimensional or 3-dimensional representation of the VOI) to the user. An input device 3050, such as a keyboard, may be coupled to processing device 3010 for communicating information and/or command selections to processing device 3010. One or more other user input devices (e.g., a mouse, a trackball or cursor direction keys) may also be used to communicate directional information, to select commands for processing device 3010 and to control cursor movements on display 3040.

It will be appreciated that treatment planning system 3000 represents only one example of a treatment planning system, which may have many different configurations and architectures, which may include more components or fewer components than treatment planning system 3000 and which may be employed with the present invention. For example, some systems often have multiple buses, such as a peripheral bus, a dedicated cache bus, etc. The treatment planning system 3000 may also include MIRIT (Medical Image Review and Import Tool) to support DICOM import (so images can be fused and targets delineated on different systems and then imported into the treatment planning system for planning and dose calculations), expanded image fusion capabilities that allow the user to treatment plan and view dose distributions on any one of various imaging modalities (e.g., MRI, CT, PET, etc.). Treatment planning systems are known in the art; accordingly, a more detailed discussion is not provided.

Treatment planning system 3000 may share its database (e.g., data stored in storage device 3030) with a treatment delivery system, such as treatment delivery system 4000, so that it may not be necessary to export from the treatment planning system prior to treatment delivery. Treatment planning system 3000 may be linked to treatment delivery system 4000 via a data link 2500, which may be a direct link, a LAN link or a WAN link as discussed above with respect to data link 1500. It should be noted that when data links 1500 and 2500 are implemented as LAN or WAN connections, any of diagnostic imaging system 2000, treatment planning system 3000 and/or treatment delivery system 4000 may be in decentralized locations such that the systems may be physically remote from each other. Alternatively, any of diagnostic imaging system 2000, treatment planning system 3000 and/or treatment delivery system 4000 may be integrated with each other in one or more systems.

Treatment delivery system 4000 includes a therapeutic and/or surgical radiation source 4010 to administer a prescribed radiation dose to a target volume in conformance with a treatment plan. Treatment delivery system 4000 may also include an imaging system 4020 to capture intra-treatment images of a patient volume (including the target volume) for registration or correlation with the diagnostic images described above in order to position the patient with respect to the radiation source. Treatment delivery system 4000 may also include a digital processing system 4030 to control radiation source 4010, imaging system 4020, and a patient support device such as a treatment couch 4040. Digital processing system 4030 may include one or more general-purpose processors (e.g., a microprocessor), special purpose processor such as a digital signal processor (DSP) or other type of device such as a controller or field programmable gate array (FPGA). Digital processing system 4030 may also include other components (not shown) such as memory, storage devices, network adapters and the like. Digital processing system 4030 may be coupled to radiation source 4010, imaging system 4020 and treatment couch 4040 by a bus 4045 or other type of control and communication interface.

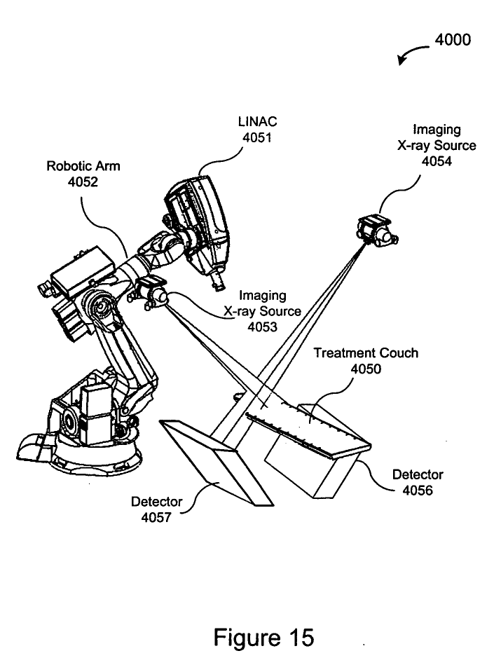

In one embodiment, as illustrated in FIG. 15, treatment delivery system 4000 may be an image-guided, robotic-based radiation treatment system (e.g., for performing radiosurgery) such as the CyberKnife® system developed by Accuray Incorporated of California. In FIG. 15, radiation source 4010 may be represented by a linear accelerator (LINAC) 4051 mounted on the end of a robotic arm 4052 having multiple (e.g., 5 or more) degrees of freedom in order to position the LINAC 4051 to irradiate a pathological anatomy (target region or volume) with beams delivered from many angles in an operating volume (e.g., a sphere) around the patient. Treatment may involve beam paths with a single isocenter (point of convergence), multiple isocenters, or with a non-isocentric approach (i.e., the beams need only intersect with the pathological target volume and do not necessarily converge on a single point, or isocenter, within the target). Treatment can be delivered in either a single session (mono-fraction) or in a small number of sessions (hypo-fractionation) as determined during treatment planning. With treatment delivery system 4000, in one embodiment, radiation beams may be delivered according to the treatment plan without fixing the patient to a rigid, external frame to register the intra-operative position of the target volume with the position of the target volume during the pre-operative treatment planning phase. The treatment delivery system 4000 can be integrated with a pathological anatomy tracking system, such as The Synchrony™ system developed by Accuray Incorporated of California, which can correlates the motion of the pathological anatomy with respiration motion in real-time, enabling the LINAC to deliver highly accurate radiation beams.

In FIG. 15, imaging system 4020 may be represented by X-ray sources 4053 and 4054 and X-ray image detectors (imagers) 4056 and 4057. In one embodiment, for example, two x-ray sources 4053 and 4054 may be nominally aligned to project imaging x-ray beams through a patient from two different angular positions (e.g., separated by 90 degrees, 45 degrees, etc.) and aimed through the patient on treatment couch 4050 toward respective detectors 4056 and 4057. In another embodiment, a single large imager can be used that would be illuminated by each x-ray imaging source. Alternatively, other numbers and configurations of imaging sources and imagers may be used.

Digital processing system 4030 may implement algorithms to register images obtained from imaging system 4020 with preoperative treatment planning images in order to align the patient on the treatment couch 4050 within the treatment delivery system 4000, and to precisely position the radiation source with respect to the target volume.

The treatment couch 4050 may be coupled to another robotic arm (not illustrated) having multiple (e.g., 5 or more) degrees of freedom. The couch arm may have five rotational degrees of freedom and one substantially vertical, linear degree of freedom. Alternatively, the couch arm may have six rotational degrees of freedom and one substantially vertical, linear degree of freedom or at least four rotational degrees of freedom. The couch arm may be vertically mounted to a column or wall, or horizontally mounted to pedestal, floor, or ceiling. Alternatively, the treatment couch 4050 may be a component of another mechanical mechanism, such as the Axum® treatment couch developed by Accuray Incorporated of California, or be another type of conventional treatment table known to those of ordinary skill in the art.

Alternatively, treatment delivery system 4000 may be another type of treatment delivery system, for example, a gantry based (isocentric) intensity modulated radiotherapy (IMRT) system. In a gantry based system, a radiation source (e.g., a LINAC) is mounted on the gantry in such a way that it rotates in a plane corresponding to an axial slice of the patient. Radiation is then delivered from several positions on the circular plane of rotation. In IMRT, the shape of the radiation beam is defined by a multi-leaf collimator that allows portions of the beam to be blocked, so that the remaining beam incident on the patient has a pre-defined shape. The resulting system generates arbitrarily shaped radiation beams that intersect each other at the isocenter to deliver a dose distribution to the target. In IMRT planning, the optimization algorithm selects subsets of the main beam and determines the amount of time that the patient should be exposed to each subset, so that the prescribed dose constraints are best met.

In other embodiments, yet another type of treatment delivery system 4000 may be used, for example, a stereotactic frame system such as the GammaKnife®, available from Elekta of Sweden. With such a system, the optimization algorithm (also referred to as a sphere packing algorithm) of the treatment plan determines the selection and dose weighting assigned to a group of beams forming isocenters in order to best meet provided dose constraints.

It should be noted that the methods and apparatus described herein are not limited to use only with medical diagnostic imaging and treatment. In alternative embodiments, the methods and apparatus herein may be used in applications outside of the medical technology field, such as industrial imaging and non-destructive testing of materials (e.g., motor blocks in the automotive industry, airframes in the aviation industry, welds in the construction industry and drill cores in the petroleum industry) and seismic surveying. In such applications, for example, “treatment” may refer generally to the application of radiation beam(s).

In the foregoing specification, the invention has been described with reference to specific exemplary embodiments thereof. It will, however, be evident that various modifications and changes may be made thereto without departing from the broader spirit and scope of the invention as set forth in the appended claims. The specification and drawings are, accordingly, to be regarded in an illustrative sense rather than a restrictive sense.

What is claimed is:

1. A method, comprising:
detecting a motion by an external reference object during respiration of a patient; and
imaging a treatment region containing a pathological anatomy and a fiducial marker positioned near the pathological anatomy to generate an internal image, wherein the external reference object is substantially non-visible on the internal image.

2. The method of claim 1, further comprising correlating the position of the first reference object with a position of a fiducial marker to track a position of the pathological anatomy in real-time.

3. The method of claim 1, wherein the internal image comprises an x-ray image generated by exposing the treatment region to an x-ray beam.

4. The method of claim 3, wherein imaging further comprises positioning the external reference object in a path of the x-ray beam.

5. The method of claim 4, wherein detecting further comprises emitting a light from the external reference object to a camera during movement by the patient.

6. The method of claim 5, wherein the external reference object comprises a fiber optic beacon connected to a breakout box via a fiber optic cable, and detecting further comprises propagating a light from the breakout box through the fiber optic cable to the fiber optic beacon.

7. The method of claim 5, wherein the external reference object comprises a light emitting diode connected to a power source via a wire, and emitting further comprises propagating an electrical current from the power source along the wire to the light emitting diode, wherein the wire has a thickness that is significantly less than a width of the fiducial marker.

8. The method of claim 3, wherein the external reference object comprises a magnetic sensor and detecting further comprises signaling the position to an interface device connected to the magnetic sensor.

9. The method of claim 8, wherein signaling further comprises generating an electromagnetic field that encompasses the magnetic sensor with a transmitter disposes near the patient.

10. The method of claim 9, wherein signaling further comprises filtering the signal to reduce jitter.

11. The method of claim 2, wherein correlating further comprises delivering a radiation treatment to the pathological anatomy captured by the internal image during respiration by the patient.

12. An apparatus, comprising:
means for detecting a motion by an external reference object during respiration of a patient; and
means for imaging a treatment region containing a pathological anatomy and a fiducial marker positioned near the pathological anatomy to generate an internal image, wherein the external reference object is substantially non-visible on the internal image.

13. The apparatus of claim 12, wherein the internal image comprises an x-ray image generated by exposing the treatment region to an x-ray beam.

14. The apparatus of claim 13, wherein means for imaging further comprises means for positioning the external reference object in a path of the x-ray beam.

15. The apparatus of claim 13, wherein the external reference object comprises a fiber optic beacon connected to a breakout box via a fiber optic cable, and means for detecting further comprises means for propagating a light from the breakout box through the fiber optic cable to the fiber optic beacon.

16. The apparatus of claim 13, wherein the external reference object comprises a light emitting diode connected to a power source via a wire, and means for detecting further comprises means for propagating an electrical current from the power source along the wire to the light emitting diode, wherein the wire has a thickness that is significantly less than a width of the fiducial marker.

17. The apparatus of claim 13, wherein the external reference object comprises a magnetic sensor and means for detecting further comprises means for signaling the position to an interface device connected to the magnetic sensor.

18. The apparatus of claim 17, wherein means for signaling further comprises means for generating an electromagnetic field that encompasses the magnetic sensor with a transmitter disposes near the patient.

19. The apparatus of claim 12, wherein means for generating further comprises means for delivering a radiation treatment to a pathological anatomy captured by the internal image during respiration by the patient.

20. A method comprising:
providing a fiber optic beacon disposed on a vest;
propagating a light through a fiber optic cable to the fiber optic beacon during a motion of the fiber optic beacon caused by respiration of a patient wearing the vest; and
detecting the light to determine the motion of the fiber optic beacon during respiration of the patient.

21. The method of claim 20, further comprising:
propagating an x-ray beam along a path from an x-ray source to a detector to generate an internal image; and
positioning the patient in the path of the x-ray beam, wherein at least a portion of the fiber optic cable or the fiber optic beacon is situated in the path of the x-ray beam, and wherein the portion of the fiber optic cable or the fiber optic beacon is non-visible in the internal image.

22. The method of claim 21, wherein propagating the light further comprises originating the light from a breakout box coupled to the fiber optic cable.

23. The method of claim 21, wherein detecting further comprises aiming a camera toward the fiber optic beacon to sense the light.

24. The method of claim 21, wherein the internal image contains a pathological anatomy and a fiducial marker within the patient, and wherein detecting further comprises correlating the motion of the fiber optic beacon with a motion of a fiducial marker to track a movement of the pathological anatomy in real-time.

25. The method of claim 23, wherein aiming further comprises flashing the light to indicate that the fiber optic beacon is aligned with the camera and maintaining the light continuously to indicate that the fiber optic beacon is not aligned with the camera.

26. A method, comprising:
providing a light emitting diode disposed on a vest;
propagating an electrical current along a wire to the light emitting diode to generate a light during a motion of the light emitting diode caused by respiration of a patient wearing the vest; and
detecting the light to determine the motion of light emitting diode during respiration of the patient.

27. The method of claim 26, further comprising:
propagating an x-ray beam along a path from an x-ray source to a detector to generate an internal image; and
positioning the patient in the path of the x-ray beam, wherein at least a portion of the wire or light emitting diode is situated in the path of the x-ray beam, and wherein the portion of the wire or the light emitting diode is non-visible in the internal image.

28. The method of claim 27, wherein propagating the electrical current further comprises originating the light from a power source coupled to the light emitting diode.

29. The method of claim 27, wherein detecting further comprises aiming a camera toward the light emitting diode to sense the light.

30. The method of claim 27, wherein the internal image contains a pathological anatomy and a fiducial marker within the patient, and wherein detecting further comprises correlating the motion of the light emitting diode with a motion of a fiducial marker to track a movement of the pathological anatomy in real-time.

31. The method of claim 30, wherein the wire has a diameter that is substantially less than a width of the fiducial marker.

32. The method of claim 30, wherein the wire is positioned in a non-overlapping manner relative to the fiducial marker.

33. The method of claim 29, wherein aiming further comprises flashing the light to indicate that the light emitting diode is aligned with the camera and maintaining the light continuously to indicate that the light emitting diode is not aligned with the camera.

34. A method, comprising:
providing a magnetic sensor disposed on a vest;
generating an electromagnetic field around the vest; and
detecting a motion of the magnetic sensor caused by respiration of a patient wearing the vest.

35. The method of claim 34, further comprising:
propagating an x-ray beam along a path from an x-ray source to a detector to generate an internal image; and
positioning the patient in the path of the x-ray beam, wherein at least a portion of the magnetic sensor is situated in the path of the x-ray beam, and wherein the portion of the magnetic sensor is non-visible in the internal image.

36. The method of claim 35, wherein detecting further comprises transmitting a signal to an interface device coupled to the magnetic sensor.

37. The method of claim 35, wherein the electromagnetic field is formed by a transmitter positioned near the patient.

38. The method of claim 35, wherein the internal image contains a pathological anatomy and a fiducial marker within the patient, and wherein detecting further comprises correlating the motion of the magnetic sensor with a motion of a fiducial marker to track a movement of the pathological anatomy in real-time.

39. An apparatus, comprising:
a vest;
a fiber optic beacon disposed on the vest; and
a fiber optic cable having a first end coupled to the fiber optic beacon and a second end coupled to a breakout box, the fiber optic cable to propagate a light from the breakout box to the fiber optic beacon during a motion of the fiber optic beacon caused by respiration of a patient wearing the vest, wherein at least a portion of the fiber optic beacon or the fiber optic cable is positioned in a path of an x-ray beam to generate an image of a treatment region within the patient, and wherein the fiber optic beacon or the fiber optic cable is substantially non-visible on the image.

40. The apparatus of claim 39, further comprising an x-ray source to originate the path of the x-ray beam toward a detector.

41. The apparatus of claim 40, wherein the light is sensed by a camera aimed toward the fiber optic beacon.

42. The apparatus of claim 40, wherein the image of the treatment region contains a pathological anatomy and a fiducial marker, and wherein the motion of the fiber optic beacon is correlated with a motion of the fiducial marker positioned within the patient to track a movement of the pathological anatomy in real-time.

43. An apparatus, comprising:
a vest;
a light emitting diode disposed on the vest; and
a wire having a first end coupled to the light emitting diode and a second end coupled to a power source, the wire to propagate an electrical current from the power source to the light emitting diode during a motion of the light emitting diode caused by respiration of a patient wearing the vest, wherein at least a portion of the light emitting diode or the wire is positioned in a path of an x-ray beam to generate an image of a treatment region within the patient, and wherein the light emitting diode or the wire is substantially non-visible on the image.

44. The apparatus of claim 43, further comprising an x-ray source to originate the path of the x-ray beam toward a detector.

45. The apparatus of claim 44, wherein the light is sensed by a camera aimed toward the light emitting diode.

46. The apparatus of claim 44, wherein the image of the treatment region contains a pathological anatomy and a fiducial marker, and wherein the motion of the light emitting diode is correlated with a motion of the fiducial marker positioned within the patient to track a movement of the pathological anatomy in real-time.

47. The apparatus of claim 46, wherein the wire has a diameter that is substantially less than a width of the fiducial marker.

48. The apparatus of claim 46, wherein the wire is positioned in a non-overlapping manner relative to the fiducial marker.

49. An apparatus, comprising:
a vest; and
a magnetic sensor disposed on the vest, the magnetic sensor to generate a positional signal within an electromagnetic field during a motion of the magnetic sensor caused by respiration of a patient wearing the vest, wherein at least a portion of the magnetic sensor is positioned in a path of an x-ray beam to generate an image of a treatment region within the patient, and wherein the magnetic sensor is substantially non-visible on the image.

50. The apparatus of claim 49, wherein the positional signal is sensed by an interface device coupled to the magnetic sensor.

51. The apparatus of claim 50, wherein the image of the treatment region contains a pathological anatomy and a fiducial marker, and wherein the motion of the magnetic sensor is correlated with a motion of the fiducial marker positioned within the patient to track a movement of the pathological anatomy in real-time.
